(19) United States
(12) Patent Application Publication
(10) Pub. No.: US 2020/0289413 A1
(43) Pub. Date: Sep. 17, 2020

(54) COMPOSITIONS USEFUL IN THE PREVENTION AND/OR TREATMENT OF DISORDERS OF THE ORAL CAVITY, UPPER AIRWAYS AND ESOPHAGUS

(71) Applicant: Indena S.P.A., Milano (IT)

(72) Inventor: Ezio Bombardelli, Gropello Cairoli (PV) (IT)

(73) Assignee: Indena S.P.A., Milano (IT)

(21) Appl. No.: 16/884,245

(22) Filed: May 27, 2020

Related U.S. Application Data
(62) Division of application No. 16/084,989, filed on Sep. 14, 2018.

(30) Foreign Application Priority Data
Mar. 18, 2016 (IT) 102016000028801

(51) Int. Cl. A61K 9/00; A61K 36/9068; A61K 9/06; A61K 47/36; A61K 9/19; A61K 9/20

(52) U.S. Cl. CPC A61K 9/0056; A61K 36/9068; A61K 9/006; A61K 9/2018; A61K 47/36; A61K 9/19; A61K 9/06

(57) ABSTRACT

The present invention relates to compositions comprising at least one film-forming agent able to adhere in a stable way to the mucosa of the mouth, esophagus and upper airways, establishing a mechanical form of protection against attack on said mucosae.

Said compositions are useful in the prevention and/or treatment of disorders of the oral cavity, esophagus and upper airways caused by bacterial and/or viral agents and/or by chemical agents.

This Non-Provisional Application is a Divisional of U.S. Ser. No. 16/084,989 filed Sep. 14, 2018, which is a U.S. national stage of PCT/EP2017/056148 filed on 15 Mar. 2017, which claims priority to and the benefit of Italian Application No. 102016000028801 filed on 18 Mar. 2016, the contents of which are incorporated herein by reference in their entireties.

### TECHNICAL FIELD OF THE INVENTION

The present invention relates to compositions comprising at least one film-forming agent able to adhere in a stable way to the mucosa of the mouth, esophagus and upper airways, and optionally at least one compound or extract obtained from a medicinal plant having antimicrobial or antiviral activity.

Said compositions are useful in the prevention and/or treatment of disorders of the oral cavity and upper airways caused by bacterial and/or viral agents, and of the esophagus.

### BACKGROUND OF THE INVENTION

Reddening, inflammation and bacterial and/or fungal infections of the throat, with plaque formation, are symptoms that commonly accompany influenza, colds and similar disorders.

The common cold and influenza, which affect both children and adults up to three times a year on average, are mainly associated with viral infections, 40% of which are caused by rhinovirus, 10% by coronavirus and a smaller proportion by adenovirus and parainfluenza virus. Although there is no specific treatment for these disorders, antihistamines, decongestants and anti-inflammatories are considered useful, because reduction of oedema alleviates pain and shortens the length of the disorder underlying the inflammation.

These disorders sometimes involve complications due to the onset of secondary bacterial infections, because the outlets of the nasal sinuses are often obstructed due to congestion of the mucosa, where pathogenic germs can easily proliferate, causing fever and localized pain. In this case, antibiotic treatment is required in addition to symptomatic treatments.

One of the aspects to be considered during epidemics is the ease of transmission of the disease through involuntary contact with carriers.

There is still a need to identify alternative products which are useful in the prevention and/or treatment of disorders of the oral cavity, esophagus and upper airways caused by bacterial and/or viral agents and/or chemical agents.

### SUMMARY OF THE INVENTION

The invention relates to compositions comprising at least one film-forming agent able to adhere in a stable way to the mucosa of the mouth, esophagus and upper airways.

The invention also relates to the use of said compositions in the prevention and/or treatment of disorders of the oral cavity, esophagus and upper airways caused by chemical agents and/or bacterial and/or viral agents.

### DETAILED DESCRIPTION OF THE INVENTION

The present invention relates to compositions comprising at least one film-forming agent able to adhere in a stable way to the mucosa of the mouth, esophagus and upper airways.

The composition can optionally contain at least one compound or extract obtained from a medicinal plant having antimicrobial or antiviral activity.

It has now surprisingly been found that compositions comprising at least one film-forming agent able to adhere in a stable way to the mucosa of the mouth, esophagus and upper airways protect against attack by a chemical agent and/or a bacterial and/or viral agent on the mucosa, thus allowing the prevention and/or treatment of disorders of the oral cavity, esophagus and upper airways caused by chemical agents and/or bacterial and/or viral agents.

The film-forming agent is selected from the group consisting of salts of hyaluronic acid, proteoglycans or alginic acid with anthocyanidins or proanthocyanidins, in free or glycosylated form; or tannins derived from procyanidins or ellagic acid incorporated in crosslinked hyaluronic or alginic acid, by the procedures reported in the examples.

The film-forming agent is made from known polymer molecules of natural or synthetic origin, by reacting them with compounds having a strong protein bond or with extracts obtained from medicinal plants containing said compounds, such as anthocyans, anthocyanidins or proanthocyanidins, in free or glycosylated form, tannins, alkaloids and flavonoids; in particular anthocyanidins, proanthocyanidin, in free or glycosylated form, and tannins.

Extracts suitable for said purpose are extracts of Vaccinium myrtillus, Vaccinium uliginosum, Punica granatum, Vitis vinifera and Aesculus ippocastanum. The extract is preferably Vaccinium myrtillus or Vaccinium uliginosum extract. More preferably it is Vaccinium uliginosum extract

According to a preferred aspect, an extract of Vaccinium myrtillus, Vaccinium uliginosum, Punica granatum, Vitis vinifera or Aesculus ippocastanum may be used in quantities ranging from 40 to 400 mg.

The polymer molecules may be hyaluronic acid and alginic acid, due to their property of generating salts with anthocyanosides and alkaloids or forming new polymers by cross-reactions able to incorporate tannic substances with a high affinity for proteins in the crosslinked structures.

With hyaluronic acid, chondroitin sulphate, keratan sulphate, sucralfate and alginates in salt form with sodium or potassium, anthocyanosides, by double exchange, give rise to new salts wherein the oxonium base bonds the carboxyls of the corresponding polymers, leaving the phenol part exposed; in vivo, the phenol part is anchored to the muciparous protein part of the mucosa of the mouth and adnexa, such as the palatine tonsils, Waldeyer's lymphatic ring, the proximal part of the esophagus and the upper airways, and partly to the phospholipid portion.

The barrier effect is so considerable due to the adhesion of the film-forming agent to the tissue, and is far greater than that obtainable by administering the compounds separately.

The film-forming agent reacts with the protein part of the secretion of the muciparous cells to form a protective and active barrier against the aggressive external pathogen.

This reduces bacterial and viral adhesion to the tissues, and the harm which can be caused to mucosa damaged and inflamed by pathogens, acidity or food.

The compositions according to the invention therefore prevent the formation of purulent plaques deriving from saprophytic infections of the oral cavity, thus avoiding the use of antibiotics, especially in infants and the elderly.

Moreover, the compositions according to the invention perform a favourable activity by cleaning the oral cavity reducing bacterial adhesion to the mucosa, with a consequent preventive effect.

The reaction between anthocyanosides and polymer can take place in water or ethanol/water mixtures; the resulting paste-like polymer is then freeze-dried. Natural acidic polymers reacted with binders that retain the polyphenols in the matrices can be advantageously used as an alternative to salts, which are not obtainable with non-cationic molecules.

As reported in the literature, hyaluronic acid and alginic acid, like some pectins, can react with crosslinkers such as divinyl sulfone (DVS), 1,4-butanediol diglycidyl ether, water-soluble carbodiimides (e.g. 1-ethyl-3-(3-dimethylaminopropyl)carbodiimide), crosslinkers with amino groups or urea to give rise to crosslinked polymers which can incorporate other substances in the matrix, in this case polyphenols, which can increase adhesion to the mucosa or other tissues.

Many of said biopolymers are normally compatible with human tissues, but various problems arise. In fact, it has been found that not all these crosslinkers possess adequate biocompatibility with body tissues, especially the mucosa, giving rise to adverse reactions. Biopolymers were therefore selected which, when combined with polyphenols, possess high biocompatibility with the mucosa and skin tissues.

The biopolymer obtainable with urea is preferred. Said crosslinked biopolymer is a sufficiently fluid, physiologically acceptable macromolecular matrix. The end product is obtainable by reacting hyaluronic acid with urea by acid catalysis, or by other methods reported in the examples.

In some cases, hyaluronic acid salified with sodium or potassium is crosslinked by reacting it with urea, and a compound characterised by possessing two amino groups bonded to a carbonyl functional group is obtained.

The hyaluronic acid or alginic acid preferably has a molecular weight ranging between 100,000 and 10,000,000 daltons, more preferably 1,000,000 daltons.

Hyaluronic or alginic acid can normally dissolve in water in amounts ranging from 0.5 to 5% w/w, and urea in amounts ranging from 0.2 to 2% w/w, using dilute mineral acids such as sulphuric or hydrochloric acid, mainly dilute sulphuric acid, for the acid catalysis.

In the case of crosslinking, the polymers are characterised by analysis of viscosity η and shear stress τ as a function of shear rate γ by methods extensively reported in the literature, or by IR determination. The procedure anyhow establishes the consistency and applicability of the polymer to the tissues to be treated.

According to a preferred aspect, the compositions according to the present invention may further comprise at least one extract obtained from a medicinal plant having antimicrobial or antiviral activity, for example able to inhibit replication of the pathogens that usually cause colds and influenza, such as an essential oil. The extract obtained from a medicinal plant having antimicrobial or antiviral activity is preferably a lipophilic extract of Zingiber officinale.

The lipophilic extract of Zingiber officinale is preferably obtained from the roots and rhizomes.

The compositions may comprise the lipophilic extract of Zingiber officinale in amounts ranging from 0.01% w/w to 1% w/w, preferably in amounts ranging from 0.1% w/w to 0.8% w/w, and even more preferably in amounts of 0.5% w/w.

The lipophilic extract of Zingiber officinale can be obtained by extraction from the roots or rhizomes with alcohols, ketones or aliphatic ethers or, preferably, with carbon dioxide under supercritical conditions as described in EP0464298 A1 (page 2 lines 1-52, and page 5 line 45 to page 6 line 7).

According to a further aspect, the compositions may comprise excipients designed to make their flavour, and consequently their administration, pleasant.

The compositions according to the invention can be prepared by well-known methods, such as those described in “Remington's Pharmaceutical Handbook”, Mack Publishing Co., N.Y., USA.

The compositions according to the invention may preferably be formulated in the form of tablets that dissolve slowly in the oral cavity, in suitable chewing gum forms, or in the form of gels to be held in the mouth, in such a way as to guarantee a reasonable time for adhesion to the mucosa so as to form a barrier against both pathogens and mechanical injuries.

A further object of the present invention is the use of compositions comprising at least one film-forming agent able to adhere in a stable way to the mucosa of the mouth, esophagus and upper airways, and optionally at least one compound or extract obtained from a medicinal plant having antimicrobial or antiviral activity, in the prevention and/or treatment of disorders of the oral cavity, esophagus and upper airways caused by chemical agents and/or bacterial and/or viral agents.

The disorders caused by chemical agents and/or bacterial and/or viral agents may be, for example, colds, influenza, oral and esophageal mucositis of various origins, damages to the esophageal mucosa induced by hyperacidity and esophageal reflux.

It has been found that by administering the compositions according to the invention, considerable, long-lasting adhesion thereof to the mucosa is obtained. A barrier effect is thus achieved that protects the oropharyngeal/esophageal tract against attack by acids and enzymes due to reflux, and reduces the extent of the above-mentioned damage. This result is very useful in view of the high percentage of individuals suffering from said disorder.

Moreover, the barrier effect keeps bacterial and viral proliferation under control, thus reducing the transmission of pathogens at oropharyngeal level.

The examples below further illustrate the invention.

### EXAMPLE 1

### Aqueous Gel Based on Salts of Alginic Acid with Anthocyanosides

| | | |
|---|---|---|
| Vaccinium myrtillus extract (36% anthocyanosides) | 400 | mg |
| Alginic acid potassium salt | 500 | mg |
| Lipophilic extract of Zingiber officinale | 15 | mg |
| Potassium aspartame | 20 | mg |
| Xylitol | 250 | mg |

| | |
|---|---|
| Water | q.s. to 100 ml |

### EXAMPLE 2

### Preparation of Orodispersible Tablets

| | | |
|---|---|---|
| Vaccinium myrtillus extract (36% anthocyanosides) | 40 | mg |
| Alginic acid potassium salt | 250 | mg |
| Mannitol | 600 | mg |
| Ammonium glycyrrhizinate | 10 | mg |
| Sodium cyclamate | 40 | mg |
| Polysorbate 80 | 5 | mg |
| Lipophilic extract of Zingiber officinale | 8 | mg |

### EXAMPLE 3

### Preparation of Orodispersible Tablets

| | | |
|---|---|---|
| Extract of Vaccinium uliginosum | 40 | mg |
| (30% cyaniding glucoside Cl) | | |
| Alginic acid potassium salt | 200 | mg |
| Mannitol | 600 | mg |
| Ammonium glycyrrhizinate | 10 | mg |
| Sodium cyclamate | 40 | mg |
| Polysorbate 80 | 5 | mg |
| Zingiber officinale lipophilic extract | 10 | mg |

### EXAMPLE 4

### Preparation of Orodispersible Tablets

| | | |
|---|---|---|
| Extract of Vaccinium uliginosum (30% cyanidin glucoside Cl) | 40 | mg |
| Hyaluronic acid potassium salt (1 million daltons) | 200 | mg |
| Xylitol | 600 | mg |
| Ammonium glycyrrhizinate | 10 | mg |
| Sodium cyclamate | 40 | mg |
| Polysorbate 80 | 5 | mg |
| Lipophilic extract of Zingiber officinale | 10 | mg |

### EXAMPLE 5

### Preparation of Crosslinked Hyaluronic Acid in the Presence of Vaccinium Uliginosum Extract Containing 30% Cyanidin-3-O-glucoside Cl

16 g of hyaluronic acid potassium salt (1 million daltons) are dissolved in 200 ml of distilled water together with 5 g of Vaccinium uliginosum extract having a 30% cyanidin-3-glucoside content and a 35% procyanidin content. A solution of 20 g of urea in 100 ml of 1.4% hydrochloric acid is added slowly to said solution. The mixture is left under stirring for 12 h at 25° C. A bright red rubbery mass forms, which can be separated from the reaction medium by centrifugation to eliminate the excess urea and salts. The gelatinous residue is freeze-dried, and the resulting product in powder form can be used to prepare gels or orodispersible tablets.

### EXAMPLE 6

### Preparation of Crosslinked Alginic Acid in the Presence of Procyanidins Obtained from Vitis Vinifera

12 g of sodium alginate (250,000 D) are dissolved in 250 ml of saline solution; 8 g of Vitis vinifera extract containing 90% procyanidins are added, and after homogenization, 20 g of urea dissolved in 200 ml of physiological saline solution are added under stirring. The mixture is treated with 20 ml of 0.5 N hydrochloric acid. The strongly gelled solution is left to stand for 12 hours. The gel is then buffered with NaOH and poured under stirring into 500 ml of ethanol. The solid is centrifuged, then collected and freeze-dried. This product can be used in solid formulations or suitably diluted to form gels.

### EXAMPLE 7

### Preparation of Crosslinked Alginic Acid in the Presence of Vaccinium Myrtillus Extract and Punica Granatum Extract

12 g of sodium alginate (250,000 D) are dissolved in 250 ml of physiological saline solution; 4 g of Punica granatum extract containing 45% punicalagin and 3 g of Vaccinium myrtillus extract containing 36% anthocyanosides are added, and after homogenisation, 20 g of urea dissolved in 200 ml of physiological saline solution are added under stirring. The mixture is treated with 20 ml of 0.5 N hydrochloric acid. The strongly gelled solution is left to stand for 12 hours. The gel is then buffered with NaOH and poured under stirring into 500 ml of ethanol. The solid is centrifuged, then collected and freeze-dried. This product can be used in solid formulations or suitably diluted to form gels.

### EXAMPLE 8

### Preparation of Orodispersible Tablets

| | | |
|---|---|---|
| Crosslinked compound obtained in example 5 | 200 | mg |
| Lipophilic extract of Zingiber officinale | 5 | mg |
| Xylitol | 400 | mg |
| Ammonium glycyrrhizinate | 10 | mg |
| Sodium cyclamate | 40 | mg |
| Polysorbate 80 | 5 | mg |

### EXAMPLE 9

### Preparation of Orodispersible Tablets

| | | |
|---|---|---|
| Crosslinked compound obtained in example 6 | 200 | mg |
| Lipophilic extract of Zingiber officinale | 5 | mg |
| Xylitol | 400 | mg |
| Ammonium glycyrrhizinate | 10 | mg |
| Sodium cyclamate | 40 | mg |
| Polysorbate 80 | 5 | mg |

1. Process for preparing compositions comprising at least one film-forming agent able to adhere in a stable manner to the mucosa of oral cavity, esophagus and upper airways, said process comprising:
cross-reacting salts of hyaluronic acid, proteoglycans or alginic acid with anthocyanidins or proanthocyanidins in free or glycosylated form, or with tannins derived from procyanidins or ellagic acid, or with extracts obtained from medicinal plants containing anthocyanidins or proanthocyanidin in free or glycosylated form, or tannins;
obtaining said crosslinked film-forming agent.

2. The process according to claim 1, wherein the hyaluronic acid, proteoglycans or alginic acid react with anthocyanosides in the presence of water or ethanol/water mixtures.

3. The process according to claim 1, wherein the cross-reaction is performed in the presence of crosslinkers selected from divinyl sulfone (DVS), 1,4-butanediol diglycidyl ether, water-soluble carbodiimides including 1-ethyl-3-(3-dimethylaminopropyl)carbodiimide, crosslinkers with amino groups or urea.

4. The process according to claim 3, wherein the crosslinker is urea.

5. The process according to claim 3, wherein the hyaluronic acid or alginic acid is reacted with urea by acid catalysis.

6. The process according to claim 5, wherein the hyaluronic acid is salified with sodium or potassium.

7. The process according to claim 5, wherein hyaluronic or alginic acid are dissolved in water in amounts ranging from 0.5 to 5% w/w, and urea in amounts ranging from 0.2 to 2% w/w, using dilute mineral acids for the acid catalysis.

8. The process according to claim 7, wherein the dilute mineral acid is sulphuric or hydrochloric acid.

9. The process according to claim 1, wherein the hyaluronic acid or alginic acid has a molecular weight ranging from 100,000 to 10,000,000 daltons.

10. The process according to claim 9, wherein the hyaluronic acid or alginic acid has a molecular weight of 1,000,000 daltons.

11. The process according to claim 1, wherein the extracts are selected from extracts of Vaccinium myrtillus, Vaccinium uliginosum, Punica granatum, Vitis vinifera and Aesculus ippocastanum.

12. The process according to claim 11, wherein the extract is Vaccinium myrtillus extract or Vaccinium uliginosum extract.

13. The process according to claim 12, wherein the extract is Vaccinium uliginosum extract.

14. The process according to claim 1, wherein the compositions further comprise at least one compound or extract obtained from a medicinal plant having an antimicrobial or antiviral activity.

15. The process according to claim 14, wherein the extract obtained from a medicinal plant having an antimicrobial or antiviral activity is a lipophilic extract of Zingiber officinale.

16. The process according to claim 15, wherein the amount of the lipophilic extract of Zingiber officinale ranges from 0.01% w/w to 1% w/w.

17. The process according to claim 16, wherein the amount of the lipophilic extract of Zingiber officinale ranges from 0.1% w/w to 0.8% w/w.

18. The process according to claim 17, wherein the amount of the lipophilic extract of Zingiber officinale is 0.5% w/w.

19. Method of treating and/or preventing with the compositions obtained according to claim 1 disorders of the oral cavity, esophagus and upper airways caused by chemical and/or bacterial and/or viral agents in humans in need thereof, said method comprising:
administering said compositions to said humans; and
treating and/or preventing said disorders.

20. The method according to claim 19, wherein the disorders caused by chemical and/or bacterial and/or viral agents are colds, influenza, oral and esophageal mucositis of various origins, and damages to the esophageal mucosa induced by hyperacidity and esophageal reflux.
